(19) United States
(12) Patent Application Publication
(10) Pub. No.: US 2008/0132968 A1
(43) Pub. Date: Jun. 5, 2008

(54) CARDIAC ACTIVITY CONTROL OF GASTRIC ELECTRICAL STIMULATOR

(75) Inventor: Warren L. Starkebaum, Plymouth, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/031,276

(22) Filed: Feb. 14, 2008

Related U.S. Application Data
(62) Division of application No. 10/836,354, filed on Apr. 30, 2004.
Application No. 10/836,354 is a continuation-in-part of application No. 09/537,070, filed on Mar. 28, 2000, now Pat. No. 6,853,862.
(60) Provisional application No. 60/168,966, filed on Dec. 3, 1999.

(51) Int. Cl. A61N 1/36

(52) U.S. Cl. 607/40

(57) ABSTRACT

Methods and systems for stimulating a gastrointestinal system and for modifying a stimulation signal based on an indicator of autonomic nervous system function are described. One indicator of autonomic nervous system described is cardiac activity, including heart rate variability. Methods for selecting candidate patients for gastrointestinal stimulation therapy based on an indicator of autonomic nervous system function and methods of treating patients at risk of or suffering from gastrointestinal disorders by modifying therapy based on an indicator of autonomic function are also discussed.

### RELATED APPLICATIONS

This application is a continuation-in-part application of U.S. patent application Ser. No. 09/537,070, filed Mar. 28, 2000, now U.S. Pat. No. 6,853,862, issued Feb. 8, 2005, which claims the benefit of priority to U.S. Provisional Application Ser. No. 60/168,966, filed Dec. 3, 1999, and is a divisional application of U.S. patent application Ser. No. 10/836,354, filed Apr. 30, 2004, which application is hereby incorporated herein by reference in its entirety.

### FIELD

This disclosure relates to medical devices and to stimulation of the digestive system. This disclosure also relates to use of cardiac activity parameters to modify output medical devices and to modify stimulation of the digestive system.

### BACKGROUND

Many patients having a dysfunction of their autonomic nervous system suffer from gastrointestinal disorders. In part this may be due to autonomic regulation of gastrointestinal organs. In theory, modulation of autonomic output may result in improvement in symptoms associated with certain gastrointestinal disorders.

Electrical stimulation of a patient's digestive system or tissue thereof may also be beneficial in the treatment of gastrointestinal disorders. Electrical stimulation of a digestive system or portion thereof has been described for treating, e.g. eating, endocrine, and motility disorders, such as obesity, gastro-esophageal reflux disease (GERD), constipation, and gastroparesis.

In addition, electrical stimulation of the digestive system can affect the autonomic nervous system and its various indicators, such heart rate variability, which can serve as a measure of autonomic nervous system function. The vagus nerve provides one possible mechanism through which digestive stimulation may affect heart rate variability, other measures of cardiac activity, or other measures of autonomic nervous system function. Because the vagus nerve innervates both the digestive organs, such as the stomach, and the heart, stimulation of the digestive system may affect cardiac activity via a vagal afferent pathway.

A relationship exists between gastrointestinal disorders, the autonomic nervous system, and electrical stimulation of the digestive system. However, to date there has been no attempt to monitor cardiac activity, such as heart rate variability, or other indicators of autonomic function to determine whether a patient suffering from a gastrointestinal disorder may respond favorably to gastroelectric stimulation therapy or to modify parameters of gastroelectric stimulation therapy.

### SUMMARY

In an embodiment, the invention provides a method for identifying a candidate patient for digestive stimulation therapy. A candidate patient may be a patient at risk of or suffering from a gastrointestinal disorder. The method comprises measuring an indicator of the patient's autonomic nervous system function, and determining whether the measured indicator is indicative of autonomic dysfunction. A patient having an indicator indicative of autonomic dysfunction may be identified as a candidate patient for electrical stimulation of the digestive system or a portion thereof. The indicator may be an indicator associated with cardiac activity.

An embodiment of the invention provides a method for modifying a parameter of digestive stimulation therapy. The method comprises applying a stimulation signal to a digestive system or portion thereof and sensing an indicator of autonomic nervous system function. The method further comprises modifying a parameter of the stimulation signal based on the sensed indicator. The indicator may be an indicator associated with cardiac activity.

In an embodiment, the invention provides a digestive stimulation system. The system comprises a pulse generator adapted to apply an electrical stimulation signal via a lead or other suitable device to a digestive system or portion thereof. The pulse generator may be implantable within a subject, such as, e.g. a patient. The system further comprises a sensor for measuring an indicator of autonomic nervous system function. The sensor may be implantable within a subject. The sensor is coupled to the pulse generator in a manner to allow modification of a stimulation signal parameter in response to the sensed event. The indicator may be an indicator associated with cardiac activity.

An embodiment of the invention provides a method for treating a patient at risk of or suffering from a gastrointestinal disorder. The method comprises applying a stimulation signal to a digestive system or portion thereof and sensing an indicator of autonomic nervous system function. The method further comprises modifying a parameter of the stimulation signal based on the sensed indicator. The modification may be designed to normalize the indicator of autonomic function. The indicator may be an-indicator associated with cardiac activity.

In an embodiment, the invention provides a computer-readable medium comprising program instructions. The program instructions cause a programmable processor to determine whether an improvement in autonomic function has occurred based on information from a sensor capable of detecting an indicator of autonomic nervous system function. The program instructions may further cause the programmable processor to instruct a pulse generator to modify a parameter of an electrical stimulation signal based on whether an improvement has occurred. A medical device may comprise the computer-readable medium.

### BRIEF DESCRIPTION OF THE DRAWINGS

These and other objects, features and advantages of the present invention will be more readily understood from the following detailed description of the preferred embodiments thereof, when considered in conjunction with the drawings, in which like reference numerals indicate identical structures throughout the several views, and wherein:

FIG. 1a illustrates one suitable arrangement for implanting one embodiment of a digestive-electric stimulation system of the present invention;

FIG. 1b shows illustrative components of one embodiment of a digestive-electric stimulation system of the present invention;

FIG. 1c shows an illustrative IPG and associated medical electrical leads according to one embodiment of the present invention;

FIG. 2a shows a block diagram of one embodiment of an open-loop digestive-electric stimulation system of the present invention;

FIG. 2b shows a block diagram of one closed-loop embodiment of a digestive-electric stimulation system of the present invention;

FIG. 2c shows a block diagram of another embodiment of a closed loop digestive-electric stimulation system of the present invention;

FIG. 2d shows a signal amplitude vs. time chart obtained in accordance with an embodiment of the present invention;

FIG. 3 shows a digestive electric stimulation system according to an embodiment of the present invention;

FIG. 4 shows a block diagram of one embodiment of the present invention;

FIG. 5a shows one embodiment of a digestive stimulation system of the present invention;

FIGS. 5b through 5f illustrate various embodiments of medical electrical leads suitable for use in various embodiments of a system of the present invention;

FIGS. 6a through 6d illustrate cross-sectional views of various portions of a patient's gastrointestinal tract and the nerve innervation associated therewith;

FIGS. 7a through 7f illustrate various electrode locations in or near the stomach and/or vagus nerve of a patient that may be stimulated and/or sensed in accordance with several embodiments of the present invention;

FIG. 8 illustrates various locations in or near the stomach and/or vagus nerve of a patient for feedback control sensors according to some embodiments of closed-loop feedback control systems of the present invention;

FIGS. 9a through 9c illustrate stimulation pulse, regime and control parameters according to some embodiments of the present invention;

FIG. 10 illustrates several methods of stimulating a patient's stomach and/or vagus nerve so as to treat a gastrointestinal disorder in a patient; and

FIG. 11 is flow chart according to an embodiment of the invention.

The drawings are not necessarily to scale.

### DETAILED DESCRIPTION

In the following description, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

According to the present invention, electrical stimulation of an appropriate portion a subject's digestive system may influence function of the subject's autonomic nervous system. As autonomic dysfunction may relate to gastrointestinal disorders, autonomic nervous system function of a subject suffering from or at risk of a gastrointestinal disorder may be monitored to determine whether electrical stimulation of a subject's digestive system may be warranted and/or effective. Thus, a subject suffering from or at risk of a gastrointestinal disorder may benefit not only from electrical stimulation of their digestive system or a portion thereof but also from modifying one or more parameter of the stimulation based on how the subject's autonomic nervous system is functioning. Various indicators of autonomic nervous system function, some of which are discussed in more detail below, may be used to determine whether the subject's autonomic nervous system is functioning properly. Based on one or more of the indicators of autonomic function, electrical stimulation of a subject's digestive system may be modified to enhance therapeutic efficacy regarding the gastrointestinal disorder while seeking to normalize the indicators of autonomic function. Further, the determination of whether a subject suffering from or at risk of a gastrointestinal disorder may benefit from electrical stimulation of their digestive system or a portion thereof can be enhanced by determining whether the subject's autonomic nervous system is functioning properly.

Regardless of the mechanism by which stimulation of a digestive system or portion thereof affects autonomic nervous system function, various embodiments of the present invention exploit the relationship between gastric stimulation and autonomic function. One mechanism by which gastric stimulation may affect autonomic function is via a vagal afferent pathway. Nerve impulses generated by stimulation of an appropriate portion of the digestive system may travel along a vagal afferent pathway to the brain and then along a vagal efferent pathway from the brain to various target organs, some of which are within the digestive system. It will be recognized that additional nerves and pathways may be involved in allowing stimulation of a portion of the digestive system to affect the autonomic nervous system.

The publications listed in Table 1 below are generally relevant to stimulation of a digestive system or portions thereof, and at least some of the devices and methods disclosed in the patents and publications cited herein may be modified advantageously in accordance with the teachings of the present invention.

TABLE 1
Publications related to gastrointestinal stimulation

Kenneth Koch et al., “An Illustrated Guide To Gastrointestinal Motility,”
Electrogastrography, 2^{nd} Ed., pp. 290-307 (1993).
Kenneth Koch et al., “Functional Disorders of the Stomach,” Seminars in Gastrointestinal
Disease, Vol. 7, No. 4, 185-195 (October 1996).
Kenneth Koch, “Gastroparesis: Diagnosis and Management,” Practical Gastroenterology
(November 1997).
Babajide Familoni et al., “Efficacy of Electrical Stimulation at Frequencies Higher than
Basal Rate in Mayine Stomach,” Digestive Diseases and Sciences, Vol. 42, No. 5 (May
1997).
Babajide O. Familoni, “Electrical Stimulation at a Frequency Higher than Basal Rate in
Human Stomach,” Digestive Diseases and Sciences, Vol. 42, No. 5 (May 1997).
Grundy, D. and Scratcherd, T., “Effects of stimulation of the vagus nerve in bursts on
gastric acid secretion and motility in the anaesthetized ferret,” J. Physiol., 333: 451-461,
1982.
Sasaki, N., et al., “Selective action of a CCK-B/gastrin receptor antagonist, S-0509, on
pentagastrin-, peptone meal- and beer-stimulated gastric acid secretion in dogs,”
Aliment. Pharmacol. Ther., 14: 479-488, 2000.
Sjodin, L., “Gastric acid responses to graded vagal stimulation in the anaesthetized cat,”
Digestion, 12(1): 17-24, 1975.
Physician's Manual, NeuroCyberonics Prosthesis, Bipolar Lead, Model 300, September,
2001.
U.S. Pat. No. 5,188,104 to Wernicke et al. for “Treatment of Eating Disorders by
Nerve Stimulation.”
U.S. Pat. No. 5,231,988 to Wernicke et al. for “Treatment of Endocrine Disorders by
Nerve Stimulation.”
U.S. Pat. No. 5,263,480 to Wernicke et al. for “Treatment of Eating Disorders by
Nerve Stimulation.”
U.S. Pat. No. 5,292,344 to Douglas for “Percutaneously placed electrical
gastrointestinal pacemaker INSy system, sensing system, and pH monitoring system,
with optional delivery port.”
U.S. Pat. No. 5,423,872 to Cigaina for “Process and Device for Treating Obesity and
Syndrome Motor Disorders of the Stomach of a Patient.”
U.S. Pat. No. 5,540,730 to Terry for “Treatment of motility disorders by nerve
stimulation.”
U.S. Pat. No. 5,690,691 to Chen for “Gastro-intestinal pacemaker having phased multi-
point stimulation.”
U.S. Pat. No. 5,716,385 to Mittal for “Crural diaphragm pacemaker and method for
treating esophageal reflux disease.”
U.S. Pat. No. 5,836,994 to Bourgeois for “Method and apparatus for electrical
stimulation of the gastrointestinal tract.”
U.S. Pat. No. 5,925,070 to King et al. for “Techniques for adjusting the locus of
excitation of electrically excitable tissue.”
U.S. Pat. No. 5,941,906 to Barreras et al. for “Implantable, modular tissue INS.”
U.S. Pat. No. 6,083,249 to Familoni for “Apparatus for sensing and stimulating
gastrointestinal tract on-demand”
U.S. Pat. No. 6,097,984 to Douglas for “System and method of stimulation for treating
gastro-esophageal reflux disease.”
U.S. Pat. No. 6,238,423 to Bardy for “Apparatus and method for treating chronic
constipation.”
U.S. Pat. No. 6,381,496 to Meadows et al. for “Parameter context switching for an
implanted device.”
U.S. Pat. No. 6,393,325 to Mann et al. for “Directional programming for implantable
electrode arrays.”
U.S. Pat. No. 6,449,511 to Mintchev for “Gastrointestinal electrical INS having a
variable electrical stimulus.”
U.S. Pat. No. 6,453,199 to Kobosev for “Electrical Gastro-Intestinal Tract INS.”
U.S. Pat. No. 6,516,227 to Meadows et al. for “Rechargeable spinal cord INS system.”
U.S. Patent Application Publication No. 2002 165589 for “Gastric Treatment and
Diagnosis Device and Method.”
U.S. Patent Application Publication No. 2003 014086 for “Method and Apparatus for
Electrical Stimulation of the Lower Esophageal Sphincter.”
U.S. Patent Application Publication No. 2002 116030 for “Electrical stimulation of the
Sympathetic Nerve Chain.”
U.S. Patent Application Publication No. 2002 193842 for “Heartburn and Reflux Disease
Treatment Apparatus.”
U.S. Patent Application Publication No. 2002 103424 for “Implantable Medical Device
Affixed Internally within the Gastrointestinal Tract.”
U.S. Patent Application Publication No. 2002 198470 for “Capsule and Method for
Treating or Diagnosing the Intestinal Tract.”
PCT Patent Application WO 0089655 for “Sub-Mucosal Gastric Implant Device and
Method.”
PCT Patent Application WO 0176690 for “Gastrointestinal Electrical Stimulation.”
PCT Patent Application WO 02087657 for “Gastric Device and Suction Assisted Method
for Implanting a Device on a Stomach Wall.”
PCT Patent Application WO 0238217 for “Implantable Neuromuscular INS for
Gastrointestinal Disorders.”

All patents, patent applications, brochures, technical papers, and the like cited herein, including those listed in Table 1 are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

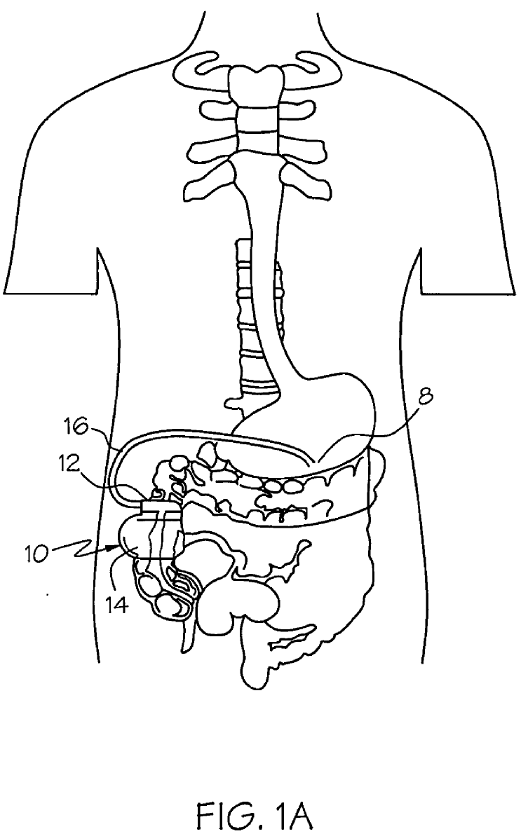

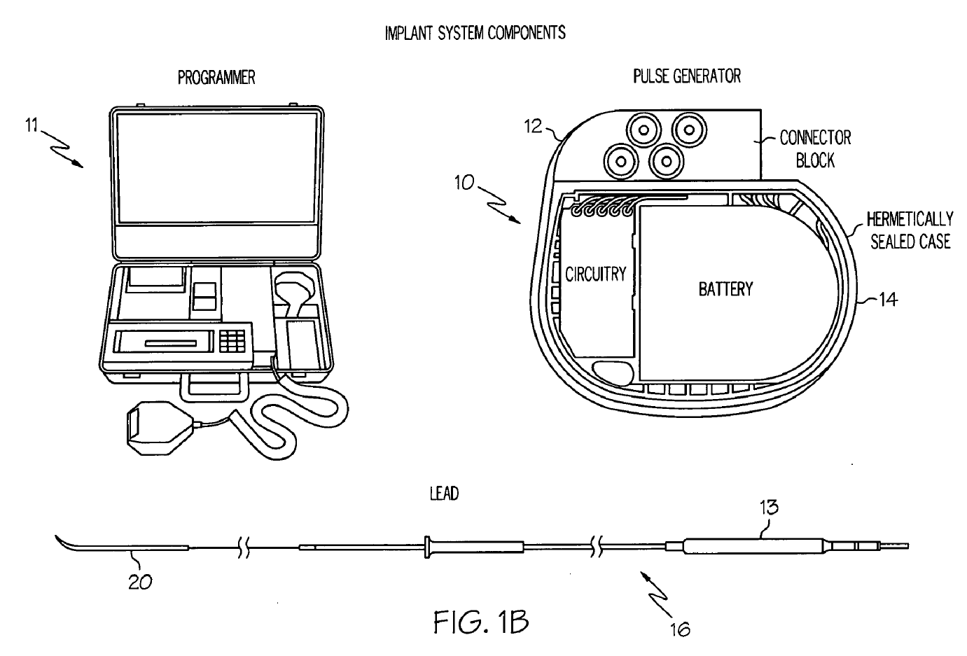

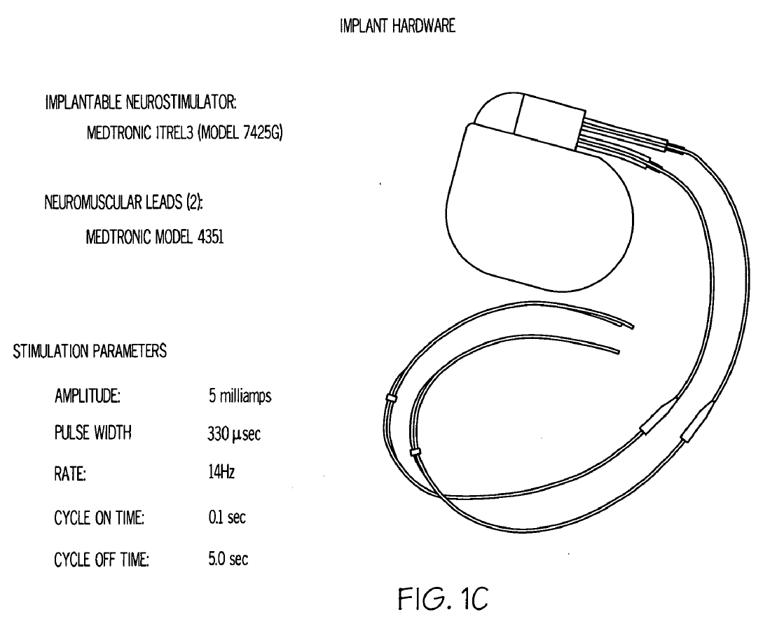

According to various embodiments of the invention, any region or combinations of regions of the digestive system may be stimulated. Preferably a stimulated region contributes to treatment of a gastrointestinal disorder. FIG. 1a shows the general environment of a gastro-electric stimulation system according to various embodiments of the invention. The patient depiction shows an abdomen and a digestive system. Included in the digestive system are a stomach, a duodenum, an intestine, a pancreas, an enteric nervous system, and a vagus nerve. While not shown, other portions of the digestive system will be readily recognized by one of skill in the art. In an embodiment, a portion of the stomach is stimulated. Various portions of the stomach are well suited for stimulation in accordance with some embodiments of the present invention. For example, the wall of the stomach is suitable for making electrical connections and the stomach is well innervated by the vagus nerve, and the stomach pacemaker region is particularly well innervated by the vagus nerve and other portions of the digestive system.

FIG. 1a further shows one embodiment of an implantable pulse generator (IPG) 10 of the present invention having a lead positioned near a desired or target tissue 8. IPG 10 shown in FIG. 1a is an implantable pulse generator system 10 comprising at least one implantable medical electrical lead 16 attached to hermetically sealed enclosure 14. Lead 16 is shown implanted at or near desired or target tissue 8. Enclosure 14 may be formed of a biocompatible material such as an appropriate metal alloy containing titanium. It is important to note that at least one more lead 18 (not shown in the drawings) may be employed in accordance with certain embodiments of the present invention, where multiple target sites are to be stimulated simultaneously or sequentially and/or where such multiple target sites are incapable of being stimulated, or are difficult to stimulate, using a single lead even if the single lead contains multiple stimulation electrodes or arrays of stimulation electrodes. FIG. 1c shows an illustrative IPG and associated medical electrical leads according to one embodiment of the present invention.

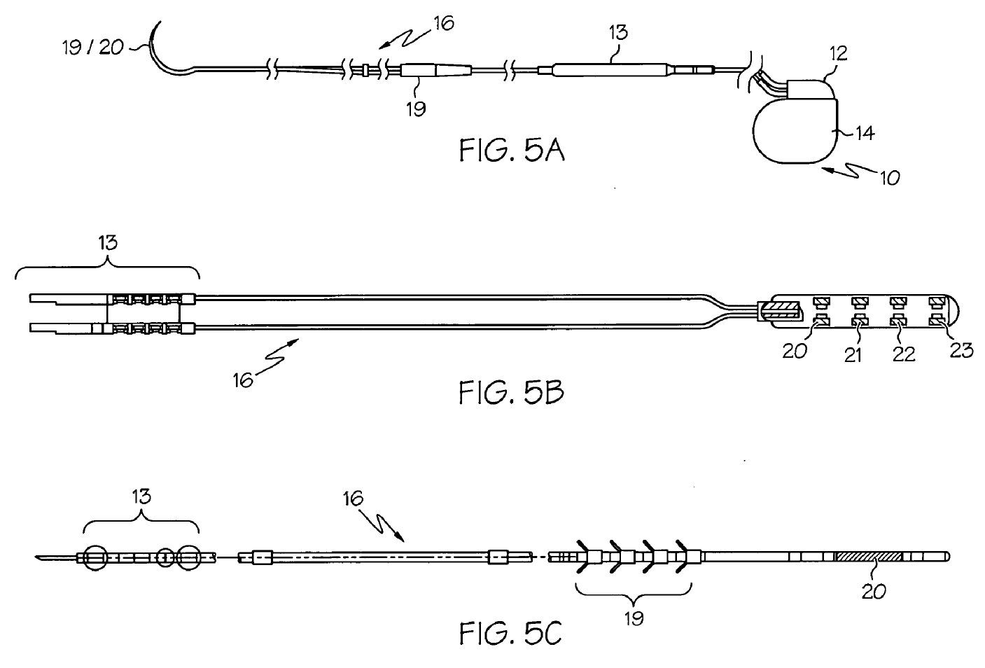

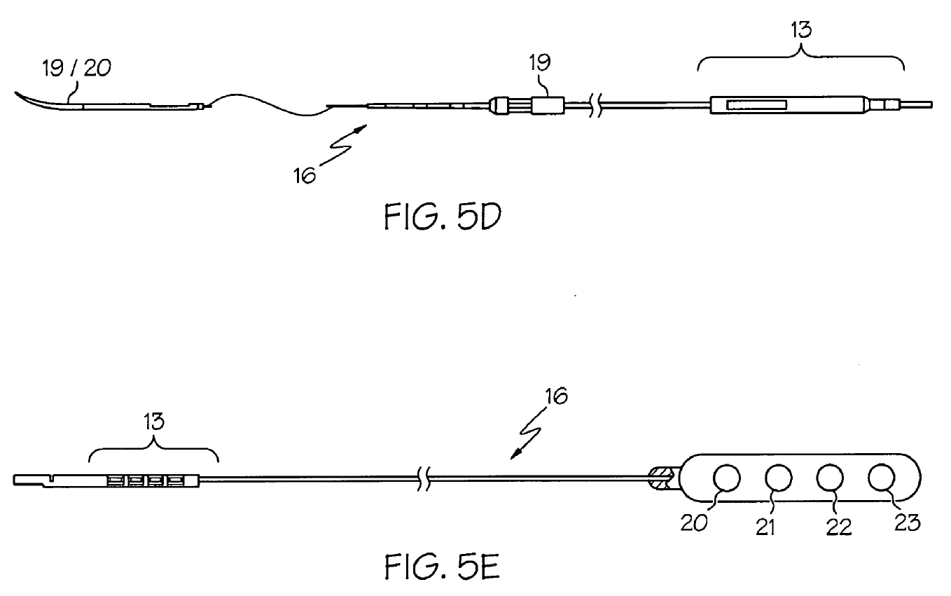

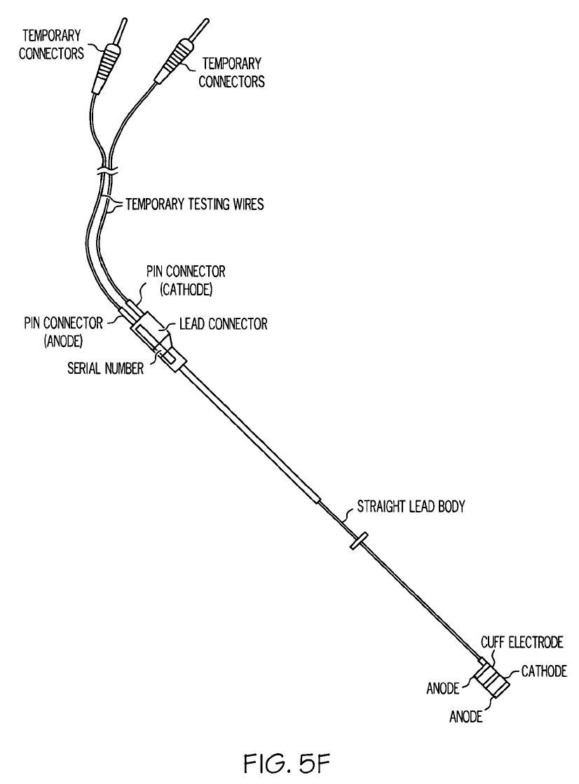

Referring now to FIG. 1b and FIGS. 5a through 5f, lead 16 provides electrical stimulation pulses to the desired target sites. Lead 16 and lead 18 may have unipolar electrodes disposed thereon (where enclosure 14 is employed as an indifferent electrode) or may have bipolar electrodes disposed thereon, where one or more electrodes disposed on a lead are employed as the indifferent electrode. In one embodiment of the present invention, lead 16 extends from lead connector 13, which in turn forms an integral portion of lead extension 15 connected at its proximal end to connector header module 12.

Leads 16 and 18 are preferably less than about 5 mm in diameter, and most preferably less than about 1.5 mm in diameter. Polyurethane is a preferred material for forming the lead body of leads 16 and 18, although other materials such as silicone may be employed. Electrical conductors extending between the proximal and distal ends of leads 16 and 18 for supplying electrical current to the electrodes are preferably formed of coiled, braided or stranded wires comprising an MP35N platinum-iridium alloy. Electrodes 20, 21, 22 and 23 may be ring electrodes, coiled electrodes, electrodes formed from portions of wire, barbs, hooks, spherically-shaped members, helically-shaped members, or may assume any of a number of different structural configurations well known in the art.

Inter-electrode distances on leads 16 and 18 are preferably about 3 mm, but other inter-electrode distances may be employed such as about 1 mm, about 2 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, about 30 mm. Preferred surface areas of electrodes 20, 21, 22 and 23 range between about 1.0 sq. mm and about 100 sq. mm, between about 2.0 sq. mm and about 50 sq. mm, and about 4.0 sq. mm and about 25 sq. mm. Preferred lengths of electrodes 20, 21, 22 and 23 range between about 0.25 mm and about 10 mm, between about 0.50 mm and about 8 mm, and about 1.0 mm and about 6 mm. Electrodes 20, 21, 22 and 23 are preferably formed of platinum, although other metals and metal alloys may be employed such as stainless steel or gold.

The distal portion of lead 16 extends to a target site 8, and is preferably held in such position by lead anchor 19. Note that lead anchor 19 may assume any of a number of different structural configurations such one or more suture sleeves, tines, barbs, hooks, a helical screw, tissue in-growth mechanisms, adhesive or glue.

One, two, three, four or more electrodes 20, 21, 22 and 23 may be disposed at the distal end of lead 16 and/or lead 18. Electrodes 20, 21, 22 and 23 are preferably arranged in an axial array, although other types of arrays may be employed such as inter-lead arrays of electrodes between the distal ends of leads 16 and 18 such that nerves or nerve portions 8 disposed between leads 16 and 18 may be stimulated. Electrode configurations, arrays and stimulation patterns and methods similar to those disclosed by Holsheimer in U.S. Pat. Nos. 6,421,566 entitled “Selective Dorsal Column Stimulation in SCS, Using Conditioning Pulses,” 5,643,330 entitled “Multichannel Apparatus for Epidural Spinal Cord Stimulation” and 5,501,703 entitled “Multichannel Apparatus for Epidural Spinal Cord INS,” the respective entireties of which are hereby incorporated by reference herein, may also be adapted or modified for use in the present invention. Electrode configurations, arrays, leads, stimulation patterns and methods similar to those disclosed by Thompson in U.S. Pat. No. 5,800,465 entitled “System and Method for Multisite Steering of Cardiac Stimuli,” the entirety of which is hereby incorporated by reference herein, may also be adapted or modified for use in the present invention to permit the steering of electrical fields. Thus, although the Figures show certain electrode configurations, other lead locations and electrode configurations are possible and contemplated in the present invention.

Leads 16 and 18 preferably range between about 4 inches and about 20 inches in length, and more particularly may be about 6 inches, about 8 inches, about 10 inches, about 12 inches, about 14 inches, about 16 inches or about 18 inches in length, depending on the location of the site to be stimulated and the distance of INS 10 from such site. Other lead lengths such as less than about 4 inches and more than about 20 inches are also contemplated in the present invention.

Typically, leads 16 and 18 are tunneled subcutaneously between the location of IPG 10 and the location or site to be stimulated. IPG 10 is typically implanted in a subcutaneous pocket formed beneath the patient's skin according to methods well known in the art. Further details concerning various methods of implanting IPG 10 and leads 16 and 18 are disclosed in the Medtronic Interstim Therapy Reference Guide published in 1999, the entirety of which is hereby incorporated by reference herein. Other methods of implanting and locating leads 16 and 18 are also contemplated in the present invention.

U.S. patent application Ser. Nos. 10/004,732 entitled “Implantable Medical Electrical Stimulation Lead Fixation Method and Apparatus” and 09/713,598 entitled “Minimally Invasive Apparatus for Implanting a Sacral Stimulation Lead” to Mamo et al., the respective entireties of which are hereby incorporated by reference herein, describe methods of percutaneously introducing leads 16 and 18 to a desired nerve stimulation site in a patient.

Some representative examples of leads 16 and 18 include MEDTRONIC nerve stimulation lead model numbers 3080, 3086, 3092, 3487, 3966 and 4350 as described in the MEDTRONIC Instruction for Use Manuals thereof, all hereby incorporated by reference herein, each in its respective entirety. Some representative examples of IPG include MEDTRONIC implantable electrical IPG model numbers 3023, 7424, 7425 and 7427 as described in the Instruction for Use Manuals thereof, all hereby incorporated by reference herein, each in its respective entirety. See also FIGS. 5b through 5f hereof, which disclose various embodiments of leads 16 and 18 suitable for use in accordance with the present invention. IPG 10 may also be constructed or operate in accordance with at least some portions of the implantable IPGs disclosed in U.S. Pat. No. 5,199,428 to Obel et al., U.S. Pat. No. 5,207,218 to Carpentier et al. or U.S. Pat. No. 5,330,507 to Schwartz, all of which are hereby incorporated by reference herein, each in its respective entirety.

Lead locations and electrode configurations other than those explicitly shown and described here are of course possible and contemplated in the present invention. Lead anchors 19 are shown in FIG. 5c as a series of tines.

A digestive electrical stimulation system may be implanted, such as with an IPG system 10, or may be located outside the patient. A programmer, separate from the digestive electrical stimulation system, may be used to modify parameters of the digestive electrical stimulation system. Programming may be accomplished with a console remote programmer such as a Model 7432 and Model 7457 memory module software or with a hand-held programmer such as an Itrel EZ, available from Medtronic, Inc. of Minneapolis, Minn.

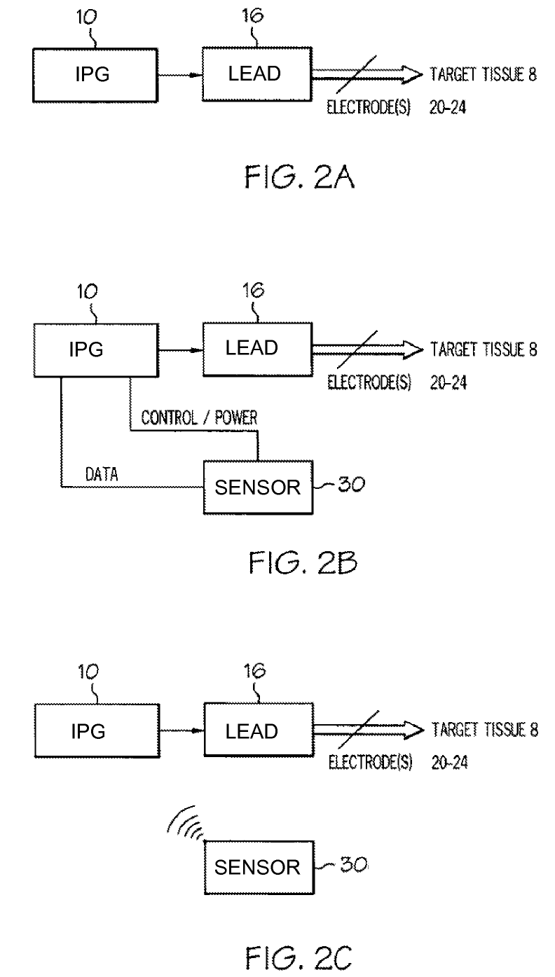

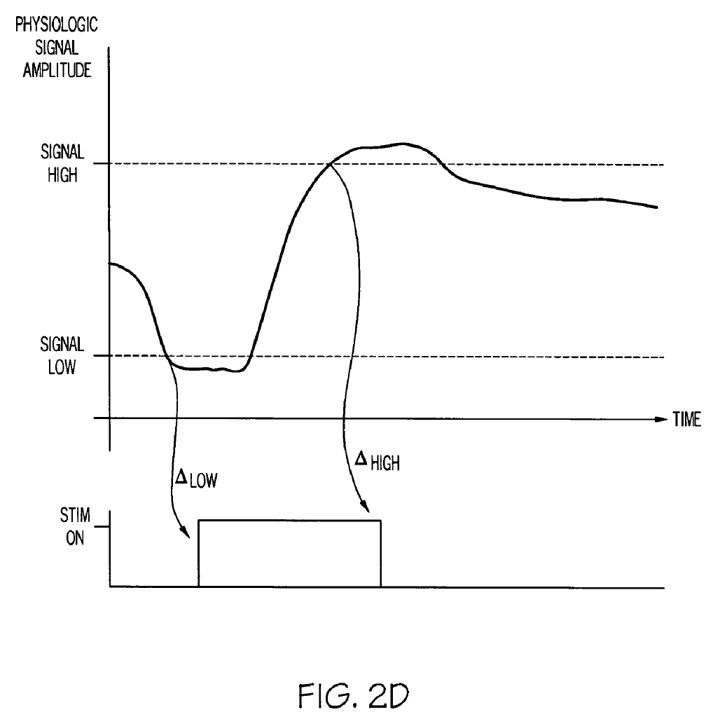

FIG. 2a shows a block diagram of one embodiment of an open-loop digestive electrical stimulation system of the present invention. FIG. 2b shows a block diagram of a closed-loop digestive electrical stimulation system. FIG. 2c shows a block diagram of yet another embodiment of a closed loop digestive electrical stimulation system of the present invention having a wireless connection between physiologic sensor 30 and IPG 10.

FIG. 2d shows an illustrative signal amplitude vs. time chart obtained in accordance with the present invention in respect of physiologic sensor 30 and the output signal generated thereby as a function of time. In such a closed-loop feedback control embodiment of the present invention, sensor 30 and sensing and computing circuitry in INS 10 cooperate to detect when a sensed signal has fallen below or risen above a predetermined threshold, as the case may be. Once the sensed signal has remained above or below the predetermined threshold for a predetermined period of time, stimulating circuitry in INS 10 is disabled. Such stimulating circuitry in INS 10 is subsequently enabled or activated when the sensed signal has once again risen above or fallen below the same or a different predetermined threshold. Similarly, stimulating circuitry in INS 10 may be enabled when the sensed signal has remained above or below the predetermined threshold for a predetermined period of time, and such circuitry may subsequently be disabled or inactivated when the sensed signal has once again risen above or fallen below the same or a different predetermined threshold.

Some examples of sensor technology that may be adapted for use in some embodiments of the present invention include those disclosed in the following U.S. patents:

U.S. Pat. No. 5,640,764 for “Method of forming a tubular feed-through
hermetic seal for an implantable medical device;”
U.S. Pat. No. 5,660,163 for “Glucose sensor assembly;”
U.S. Pat. No. 5,750,926 for “Hermetically sealed electrical feedthrough
for use with implantable electronic devices;”
U.S. Pat. No. 5,791,344 for “Patient monitoring system;”
U.S. Pat. No. 5,917,346 for “Low power current to frequency converter
circuit for use in implantable sensors;”
U.S. Pat. No. 5,957,958 for “Implantable electrode arrays;”
U.S. Pat. No. 5,999,848 for “Daisy chainable sensors and stimulators for
implantation in living tissue;”
U.S. Pat. No. 6,043,437 for “Alumina insulation for coating implantable
components and other microminiature devices;”
U.S. Pat. No. 6,088,608 for “Electrochemical sensor and integrity tests
therefor;”
U.S. Pat. No. 6,259,937 for “Implantable substrate sensor.”

In various embodiments of the present invention, sensor 30 may detect the presence and/or amount of an indicator of autonomic nervous system function. Indicators of autonomic nervous system function include cardiac activity including electrical activity of the heart such as P, Q, R, S, T amplitude, frequency and/or duration (Q-T interval, R-R interval, R to P ratio, etc); a neurotransmitter released from a parasympathetic neuron, such as acetylcholine; a neurotransmitter released from a sympathetic neuron, such as norepinephrine and/or its metabolites. Such indicators may be detected and/or measured by any suitable sensor 30. Physiologic sensor 30 may be any of a number of suitable sensor types capable of sensing an indicator of autonomic nervous system function. Examples of sensors capable of detecting cardiac activity and digital signal processing to interpret sensed cardiac activity that may be used according the teachings of the present invention include those disclosed in the patents in Table 2:

TABLE 2
Patents related to sensing and processing cardiac activity

U.S. Pat. No. 5,205,283 to Olson for “Method and apparatus for
tachyarrhythmia detection and treatment;”
U.S. Pat. No. 5,257,621 to Bardy et al. for “Apparatus for detection
of and discrimination between tachycardia and fibrillation and for
treatment of both;”
U.S. Pat. No. 5,342,402 to Olsen et al. for “Method and apparatus for
detection and treatment of tachycardia and fibrillation;”
U.S. Pat. No. 6,259,947 to Olson et al. for “Prioritized rule based method
and apparatus for diagnosis and treatment of arrhythmias;”
U.S. Pat. No. 6,556,859 to Wohlgemuth et al. for “System and method for
classifying sensed atrial events in a cardiac pacing system;” and
U.S. Pat. No. 6,029,087 to Wohlgemuth for “Cardiac pacing system with
improved physiological event classification based on DSP.”

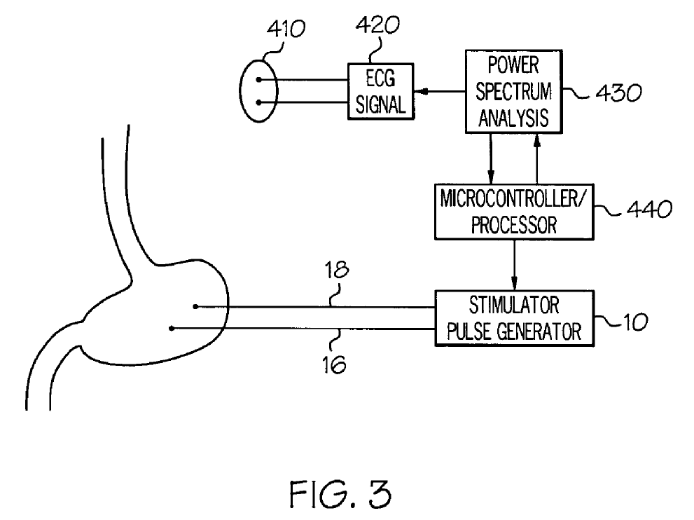

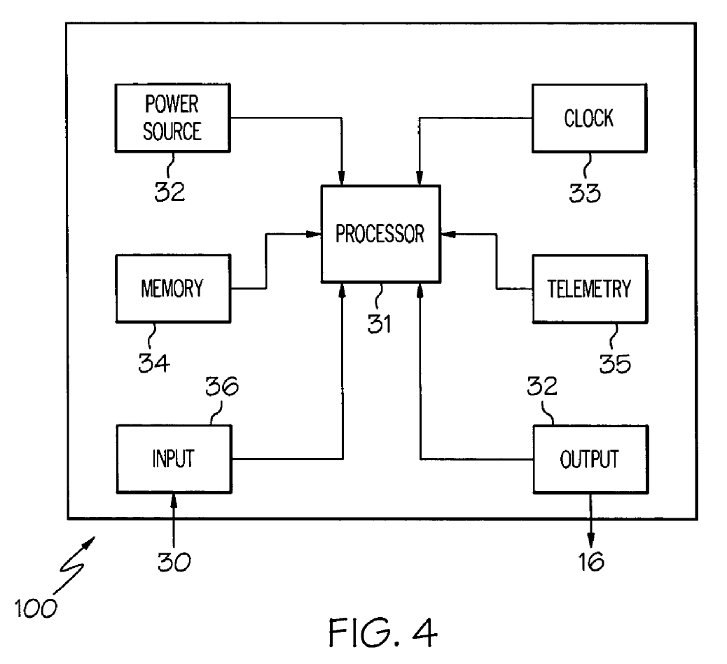

As shown in FIG. 3, cardiac activity may be measured and used to control output of an electro-stimulation therapy. While FIG. 3 depicts an embodiment related to cardiac activity, it will be understood that any indicator of autonomic function or combinations thereof may be substituted for cardiac activity or heart rate variability as discussed below. As shown in FIG. 3, one or more electrodes 410 (shown in FIG. 3 as a pair of electrodes) may be placed on or near a subject's heart to detect electrical activity of the heart (depicted as ECG signal 420 in FIG. 3). The ECG signal may be processed as discussed in, e.g., the patents listed in Table 2. Heart rate variability (periodic variation in R-R intervals; i.e., the beat-to-beat fluctuation in sinus rhythm), may also be determined. The electrodes 410 may be coupled to leads 415, which may carry signals of heart electrical activity to a power spectrum analyzer 430. Power spectrum analyzer 430 may analyze heart rate from the measured electrical activity. Power spectrum analysis may include low frequency (LF), typically between about 0.04 Hz and about 0.15 Hz, and high frequency (HF), typically between about 0.18 Hz and about 0.4 Hz, portions of the spectrum. Power spectrum analyzer 430 may be connected to microcontroller/processor 440, which may be processor 31 as discussed with regard to, e.g., FIG. 4. Microcontroller/processor 440, may be processor 31 as shown in FIG. 4 discussed below. Microcontroller/processor 440 may adjust stimulation parameters of an implantable pulse generator system. Stimulation parameters that may be adjusted include, e.g., amplitude, pulse width, pulse frequency, cycle ON, and cycle OFF times. The microcontroller/processor 440 may make adjustments of the stimulation parameters based on the LF, HF, or LF/HF from the power spectrum analysis. The microcontroller/processor 440 may make adjustments to stimulation parameters until appropriate LF, HF, and/or LF/HF values are achieved. In an embodiment, stimulation parameters may be varied as follows:

| | Parameter | Change |
|---|---|---|
| | Amplitude | Increase or decrease |
| And/or | Pulse width | Increase or decrease |
| And/or | Frequency | Increase or decrease |
| And/or | Cycle ON | Increase or decrease |
| And/or | Cycle OFF | Increase or decrease |
| And/or | Cycle ON/OFF | Increase or decrease |

Microcontroller/processor 440 may be distinct from processor 31 as described with regard to, e.g., FIG. 4. In such situations, microcontroller/processor 440 may be operably coupled to processor 31, which may control output of pulse generator 10. Microcontroller/processor 440 may be implanted in a patient or may be external to a patient. When implantable, microcontroller/processor 440 may be housed within hermetically sealed enclosure 14. When external, microcontroller/processor 440 may communicate with processor through telemetric or other wireless means. A programmer unit 11, as depicted in e.g. FIG. 1b, may comprise microcontroller/processor 440.

While the preceding discussion related to power spectrum analysis of heart rate data, it will be understood that heart rate variability (HRV) may be determined by any known or future developed technique. For example, HRV may be measured in either the time domain or the frequency domain. In the time domain, various mathematical manipulations of the R-R interval may be made in accordance with the invention. Essentially any mathematical manipulation providing meaningful HRV information may be employed. Non-limiting examples of mathematical manipulations that may be used include standard deviation of the average R-R intervals (SDNN index); standard deviations of the normal mean R-R interval obtained from successive time intervals, e.g. 5-minute periods, over 24-hour Holter recordings (SDANN index); the number of instances per hour in which two consecutive R-R intervals differ by more than about 50 msec over 24-hours (pNN50 index); the root-mean square of the difference of successive R-R intervals (rMSSD index); the difference between the shortest R-R interval during inspiration and the longest during expiration (the MAX-MIN, or peak-valley quantification of HRV); the standard deviation of successive differences of R-R intervals (SDSD), and the base of the triangular area under the main peak of the R-R interval frequency distribution diagram obtained from 24-hour recording; and the like. See Heart Rate Variability, John D. and Catherine T. MacArthus Research Network on Socioeconomic Status and Health, 5 Dec. 2001, for further discussion. The SDNN index is considered to reflect both the sympathetic and parasympathetic influence on HRV, while the other measures described above are considered to reflect cardiac parasympathetic activity. See, e.g., Jokinen (2003), “Longitudinal changes and prognostic significance of cardiovascular autonomic regulation assessed by heart rate variability and analysis on non-linear heart rate dynamics”, Academic dissertation, Department of Internal Medicine, University of Oulu. In the frequency domain, spectral analysis may be employed to determine frequency specific fluctuations of heart rate. The heart rate signal is decomposed into its frequency components (power) and quantified in terms of their relative intensities, and can be displayed as the magnitude of variability as a function of frequency (power spectrum). See, e.g., Jokinen. The total power of a signal, integrated over all frequencies, is equal to the variance of the entire signal. Typically, the area under the curve of an ultra low frequency range (typically less than about 0.003 Hz), very low frequency range (typically from about 0.003 Hz to about 0.04 Hz), low frequency range (typically from about 0.04 Hz to about 0.15 Hz) high frequency range (typically from about 0.18 Hz to about 0.4 Hz) are obtained. The high frequency range corresponds to the respiratory sinus arrhythmia. The low frequency range corresponds to vagus and cardiac sympathetic nerve activity. The ratio of low-to-high frequency spectra may serve as an index of parasympathetic-sympathetic balance. See, e.g., Heart Rate Variability, and Jokinen.

In accordance with various embodiments of the invention, heart rate variability (HRV) may be determined over any period of time suitable to determine whether the measured HRV is within a desired range or to determine if heart rate variability has increased or decreased. For example, HRV may be determined over a period of about 24 hr, about 18 hr, or about 12 hr. Of course, the time over which HRV is determined may vary from one determination of HRV to another. Further, HRV may be determined as cardiac activity is sensed, essentially on the fly, determined based on e.g. about 10 seconds worth of cardiac activity, about 30 seconds worth of cardiac activity, about 60 seconds of cardiac activity, about 5 minutes of cardiac activity, etc.

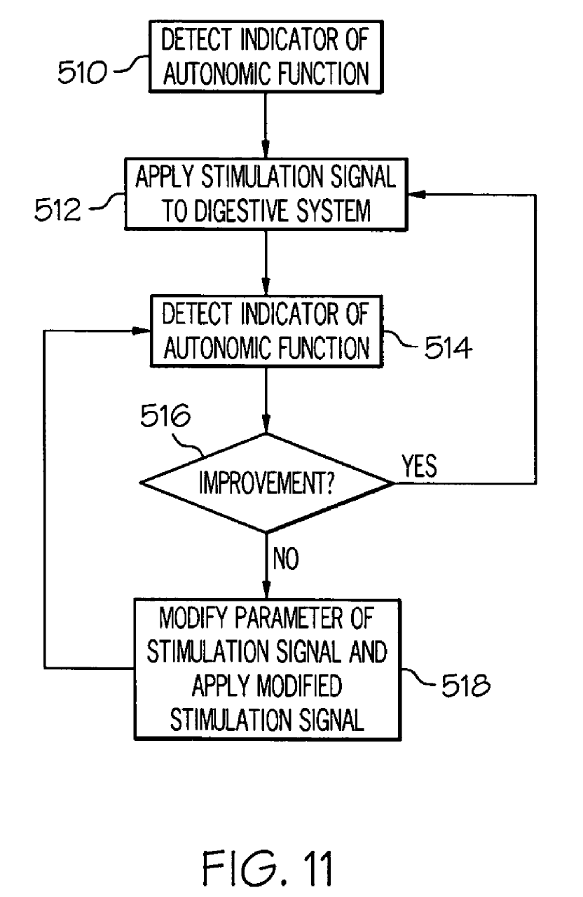

Referring to FIG. 11, a flow chart illustrating an embodiment of the invention is shown. An indicator of autonomic function, such as cardiac activity, is detected (510). Detection (510) may occur via a sensor 30. Of course detection (510) may occur via any suitable means, such a physician's diagnosis based on observation, lab results, etc. A stimulation signal is applied to a digestive system, or portion thereof (512). The indicator of autonomic function is again detected (514). A determination is then made as to whether an improvement (516) has occurred regarding autonomic function (516), based on the detected indicator. To determine whether an improvement has occurred, a detected indicator may be compared to a previously detected indicator or series of indicators. An improvement (516) may be in the form of a shortened Q-T interval, decreases in other arrhythmias, increased HRV, etc. If an improvement (516) has occurred, therapy with the previously applied stimulation parameters may be continued. If an improvement (516) has not occurred, a parameter of the stimulation signal may be modified and the modified stimulation signal may be applied to the digestive system or portion thereof (518). The indicator of autonomic function may then be detected (514) and a determination may then be made as to whether an improvement has occurred (516). The stimulation parameters are preferably modified such that the value of the detected indicator is moved towards a desired value or range. The desired value or range is preferably a value or range associated with a “normal” person, i.e., a person not suffering from dysfunction of the autonomic nervous system. Such normal values and ranges are known to those of skill in the art. Such normal values and ranges may be obtained by measuring one or more indicator of autonomic function with a population of people not suffering from a dysfunction of the autonomic nervous system and determining as normal those values and ranges that are within about, e.g., one standard deviation from the population mean.

FIG. 4 shows a block diagram illustrating some of the constituent components of IPG 10 in accordance with one embodiment of the present invention, where IPG 10 has a microprocessor-based architecture. Other architectures of IPG 10 are of course contemplated in the present invention, such as the logic or state machine architecture employed in the Medtronic Model Number 3023 INS. For the sake of convenience, those components discussed above associated with FIG. 3 and other similar components, are not shown in FIG. 4, but it should be understood that such components may be included in an IPG 10 according to various embodiments of the invention. Further for the sake of convenience, IPG 10 in FIG. 4 is shown with only one lead 16 connected thereto; similar circuitry and connections not shown in FIG. 2 apply generally to lead 18 and other additional leads not shown in the drawings. IPG 10 in FIG. 4 is most preferably programmable by means of external programming unit 11 shown in FIG. 1b. One such programmer is the commercially available Medtronic Model No. 7432 programmer, which is microprocessor-based and provides a series of encoded signals to IPG 10, typically through a programming head which transmits or telemeters radio-frequency (RF) encoded signals to IPG 10. Another suitable programmer is the commercially available Medtronic Model No. 8840 programmer, which is also microprocessor-based but features a touch control screen. Any of a number of suitable programming and telemetry methodologies known in the art may be employed so long as the desired information is transmitted to and from the implantable electrical IPG 10.

As shown in FIG. 4, IPG 10 receives input signals via sensor 30 and delivers output stimulation signals to lead 16. IPG 10 most preferably comprises a CPU, processor, controller or micro-processor 31, power source 32 (most preferably a primary or secondary battery), clock 33, memory 34, telemetry circuitry 35, input 36 and output 37. Electrical components shown in FIG. 4 may be powered by an appropriate implantable primary (i.e., non-rechargeable) battery power source 32 or secondary (i.e., rechargeable) battery power source 32. IPG 10 may also contain a battery or capacitor which receives power from outside the body by inductive coupling between an external transmitter and an implanted receiver. For the sake of clarity, the coupling of power source 32 to the various components of IPG 10 is not shown in the Figures. An antenna is connected to processor 31 via a digital controller/timer circuit and data communication bus to permit uplink/downlink telemetry through RF transmitter and receiver telemetry unit 35. By way of example, telemetry unit 35 may correspond to that disclosed in U.S. Pat. No. 4,566,063 issued to Thompson et al. It is generally preferred that the particular programming and telemetry scheme selected permit the entry and storage of electrical stimulation parameters. The specific embodiments of the antenna and other telemetry circuitry presented herein are shown for illustrative purposes only, and are not intended to limit the scope of the present invention.

An output pulse generator provides pacing stimuli to the desired target location of the digestive system through, for example, a coupling capacitor in response to a trigger signal provided by a digital controller/timer circuit, when an externally transmitted stimulation command is received, or when a response to other stored commands is received. By way of example, an output amplifier of the present invention may correspond generally to an output amplifier disclosed in U.S. Pat. No. 4,476,868 to Thompson, hereby incorporated by reference herein in its entirety. The specific embodiments of such an output amplifier are presented for illustrative purposes only, and are not intended to be limiting in respect of the scope of the present invention. The specific embodiments of such circuits may not be critical to practicing some embodiments of the present invention so long as they provide means for generating an appropriate train of stimulating pulses to the desired target location.

In various embodiments of the present invention, IPG 10 may be programmably configured to operate so that it varies the rate at which it delivers stimulating pulses to the desired target location 8 in response to one or more selected outputs being generated. IPS 10 may further be programmably configured to operate so that it may vary the morphology of the stimulating pulses it delivers. Numerous implantable electrical IPG features and functions not explicitly mentioned herein may be incorporated into IPG 10 while remaining within the scope of the present invention. Various embodiments of the present invention may be practiced in conjunction with one, two, three or more leads, or in conjunction with one, two, three, four or more electrodes.

It is important to note that leadless embodiments of the present invention are also contemplated, where one or more stimulation and/or sensing electrode capsules or modules are implanted at or near a desired target tissue site, and the capsules or modules deliver electrical stimuli directly to the site using a preprogrammed stimulation regime, and/or the capsules or modules sense electrical or other pertinent signals. Such capsules or modules are preferably powered by rechargeable batteries that may be recharged by an external battery charger using well-known inductive coil or antenna recharging means, and preferably contain electronic circuitry sufficient to permit telemetric communication with a programmer, to deliver electrical stimuli and/or sense electrical or other signals, and to store and execute instructions or data received from the programmer. Examples of methods and devices that may be adapted for use in the wireless devices and methods of the present invention include those described in U.S. Pat. No. 6,208,894 to Schulman et al. entitled “System of implantable devices for monitoring and/or affecting body parameters;” U.S. Pat. No. 5,876,425 to Schulman et al. entitled “Power control loop for implantable tissue stimulator;” U.S. Pat. No. 5,957,958 to Schulman et al. entitled “Implantable electrode arrays;” and U.S. patent application Ser. No. 09/030,106 filed Feb. 25, 1998 to Schulman et al. entitled “Battery-Powered Patient Implantable Device,” all of which are hereby incorporated by reference herein, each in its respective entirety.

FIG. 5a illustrates one embodiment of an implantable digestive-electric stimulation system suitable for use in the present invention, where the system comprises IPG 10 and at least one associated medical electrical lead 16. IPG 10 may be an implantable pulse generator such as a MEDTRONIC ITREL® 3 Model 7425 IPG that produces or generates an electrical stimulation signals adapted for the purposes of the present invention. IPG 10 may be surgically implanted such as in a subcutaneous pocket in the abdomen or positioned outside the patient. When positioned outside the patient, the IPG 10 may be attached to the patient. IPG 10 may be programmed to modify parameters of the delivered electrical stimulation signal such as frequency, amplitude, and pulse width in accordance with various embodiments of the present invention. By way of example, one or more leads 16 and 18 may be implanted into the muscle wall of the stomach such that lead electrodes 20 through 24 of adjacent leads are between about 0.5 cm apart to about 10.0 cm apart, and may be located proximal to the plexus where the vagus nerve joins the stomach.

FIGS. 5b through 5f show various embodiments of the distal end of lead 16 of the present invention. In FIGS. 5b and 5e, lead 16 is a paddle lead where electrodes 20-23 are arranged along an outwardly facing planar surface. Such a paddle lead is preferably employed to stimulate peripheral nerves. In FIG. 5c, lead 16 is a conventional quadrapolar lead having no pre-attached anchoring mechanism where electrodes 20-23 are cylindrical in shape and extend around the circumference of the lead body. In FIG. 5d, lead 16 is a quadrapolar lead having tined lead anchors. The tines may be formed from flexible or rigid biocompatible materials in accordance with the application at hand. Representative examples of some tined and other types of leads suitable, adaptable or modifiable for use in conjunction with the systems, methods and devices of the present invention include those disclosed in U.S. patent application Ser. Nos. 10/004,732 entitled “Implantable Medical Electrical Stimulation Lead Fixation Method and Apparatus” and 09/713,598 entitled “Minimally Invasive Apparatus for Implanting a Sacral Stimulation Lead” to Mamo et al., and those disclosed in U.S. Pat. No. 3,902,501 to Citron entitled “Endocardial Lead,” U.S. Pat. No. 4,106,512 to Bisping entitled “Transvenously Implantable Lead,” and U.S. Pat. No. 5,300,107 to Stokes entitled “Universal Tined Myocardial Pacing Lead.” In FIG. 5d, lead 16 is a quadrapolar lead having a pre-attached suture anchor. In FIG. 5e, lead 16 comprises needle anchor/electrode 19/20 disposed at its distal end and suture anchor 19. FIG. 5f shows lead 16 as a tri-polar cuff electrode, where cuff/anchor 19 is wrapped around desired nerve or nerve portion 8 to thereby secure the distal end of lead 16 to the nerve and position electrodes 20-22 against or near nerve or nerve portion 8. The Medtronic Model No. 3995 cuff electrode lead is one example of a lead that may be adapted for use in the present invention, the Instructions for Use manual of which entitled “INTERSTIM Manual: Model 3995 Implantable bipolar peripheral nerve and spinal root stimulation lead” is hereby incorporated by reference herein in its entirety.

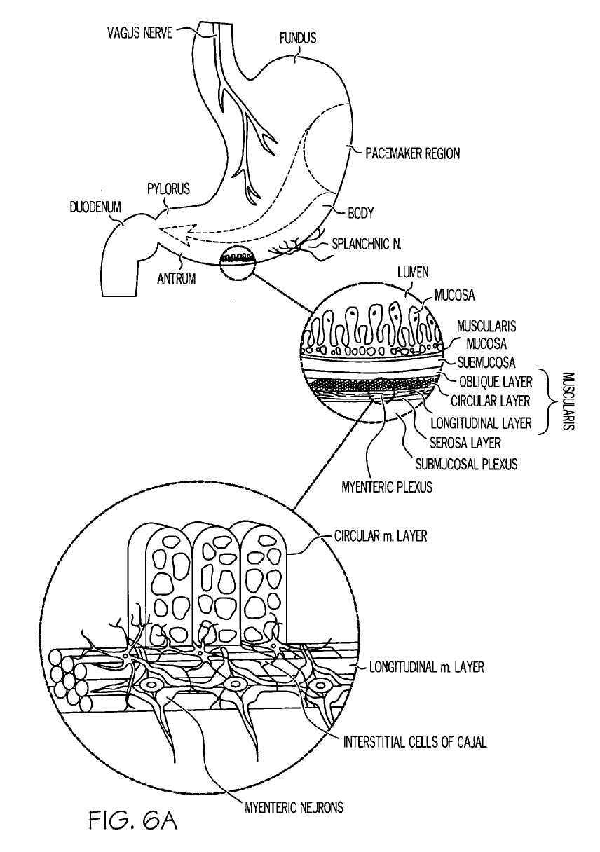

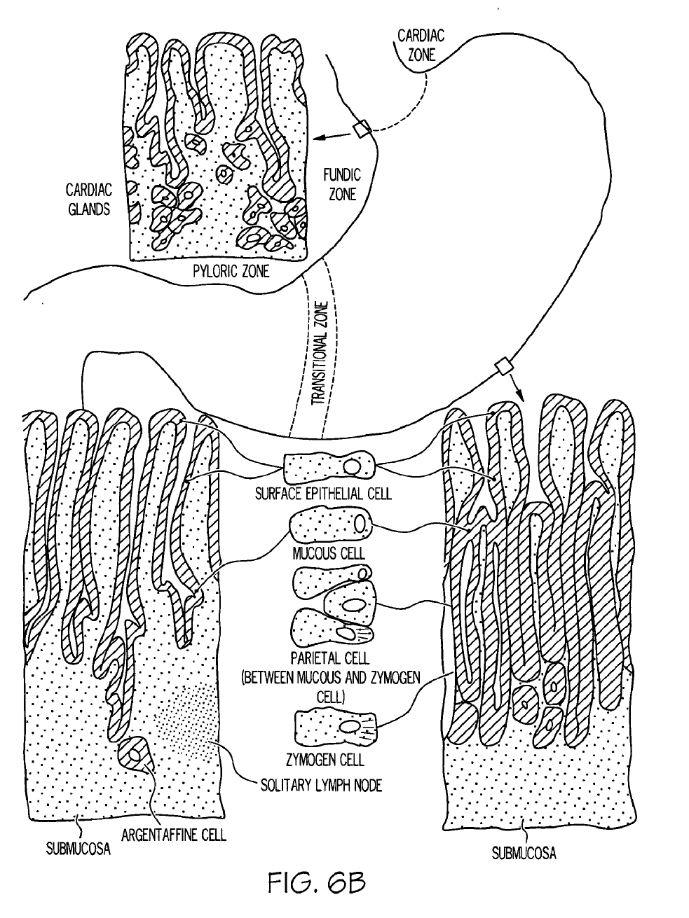

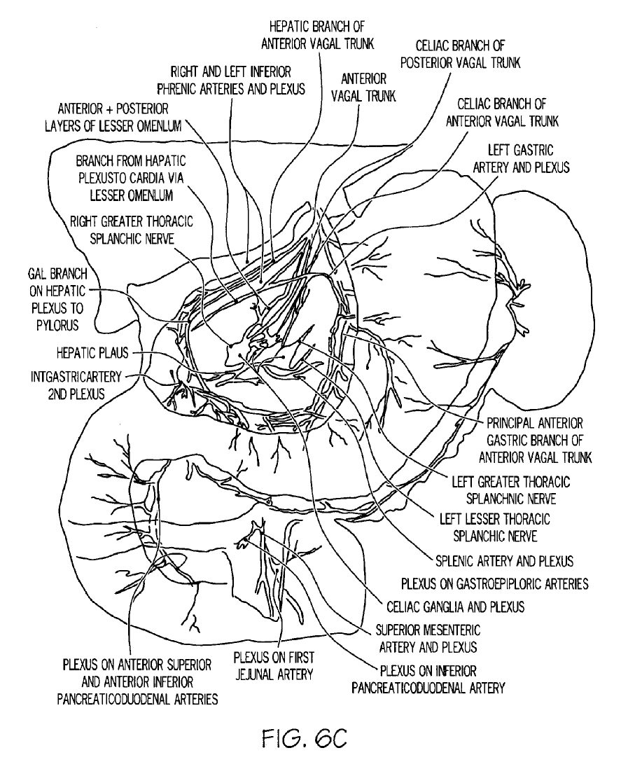

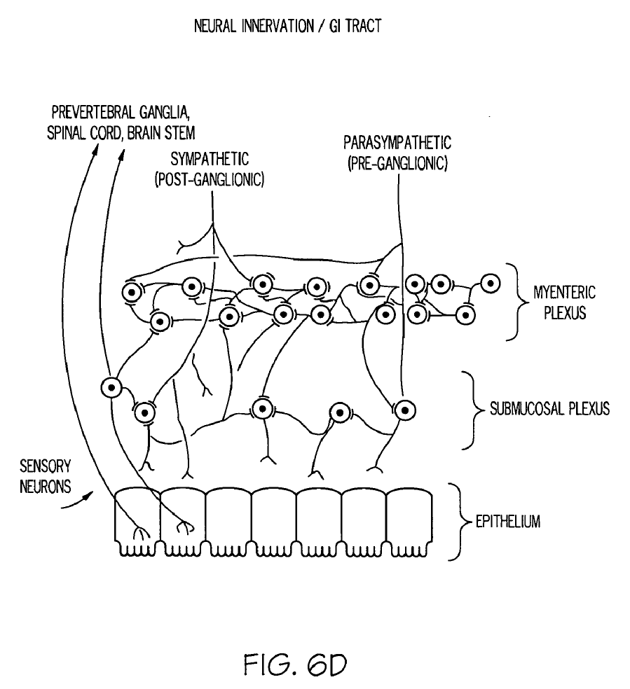

FIGS. 6a through 6d illustrate representative cross-sectional views of gross and microscopic portions of a patient's stomach to which a stimulation signal may be applied according to various embodiments of the invention. The proximal stomach is the fundus and the distal stomach is the body and antrum. The pyloric sphincter joins the antrum and the duodenum. Parasympathetic input to the stomach is supplied by the vagus nerve and the sympathetic nervous system innervates the stomach through the splanchnic nerves. On the greater curvature of the stomach between the fundus and the body is the general region of the pacemaker of the stomach. A telescoped and cross-sectional view of the antrum is shown in the circle in the middle of FIG. 5a. This view shows the gastric wall with the mucosal layer and the muscularis. The outermost muscle layer is the longitudinal layer; and running perpendicular to the longitudinal muscle layer is the circular muscle layer. There is also an oblique muscle layer in the stomach. Between the circular muscle and longitudinal muscle layers are neurons of the myenteric plexus and the enteric nervous system. The second telescoped view shown in the lower circle illustrates the anatomic proximities of the myenteric neurons and the interstitial cells of Cajal in the myenteric region between the circular and longitudinal muscle layers. The processes of the interstitial cells interdigitate with circular muscle fibers and the myenteric neurons. The interstitial cells in the myenteric plexus area are thought to be responsible for generation of slow waves or pacesetter potentials. The interstitial cells are also found in the submucosal layers, the deep musculatures plexus, and the intramuscular layers of the stomach. Leads 16 and 18 and electrodes 20-24 may be implanted in or in the vicinity of any one or more of the serosa layer, the myenteric plexus, the submucosal plexus, or any of the various layers of the muscularis (i.e., the oblique, circular or longitudinal layers).

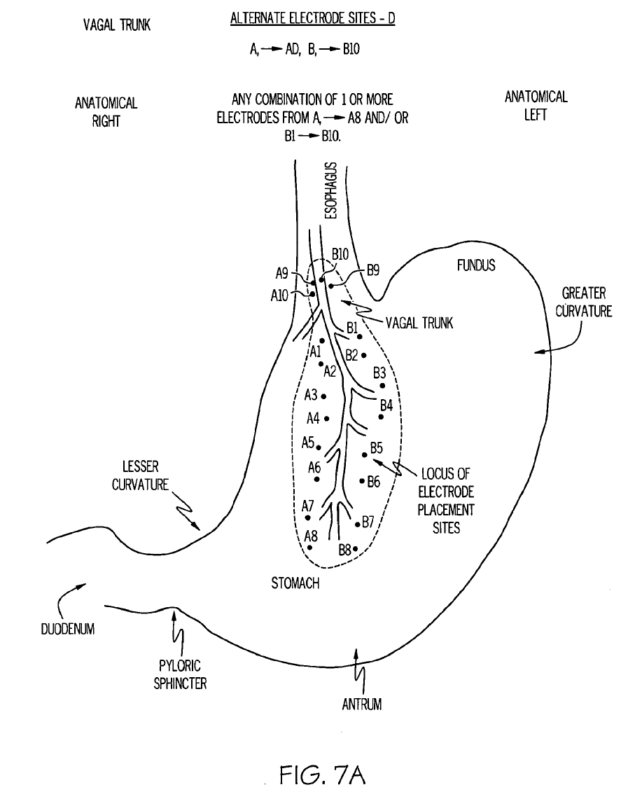

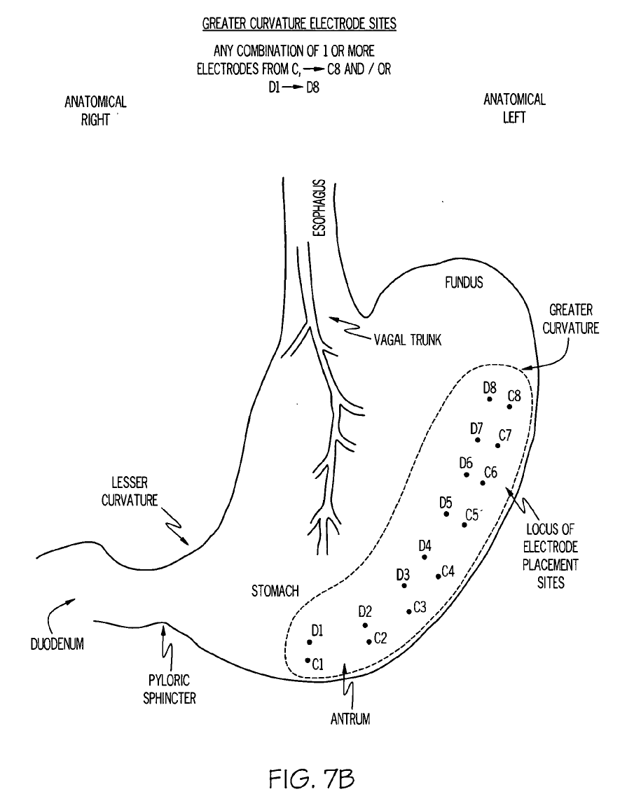

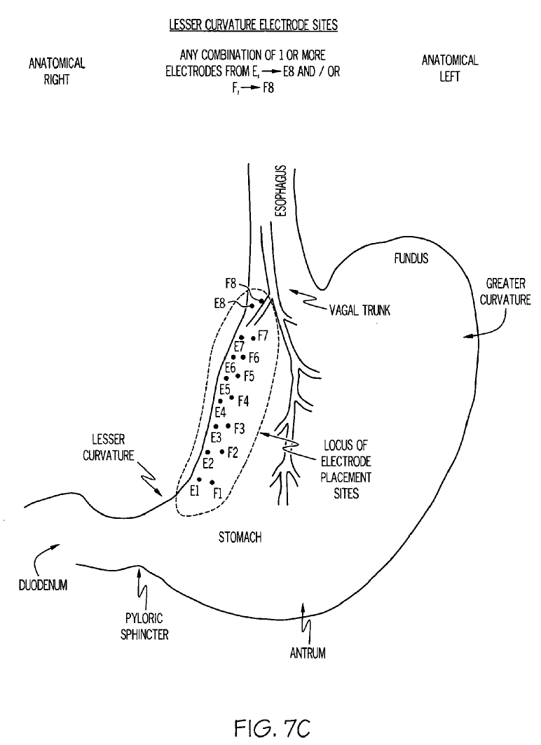

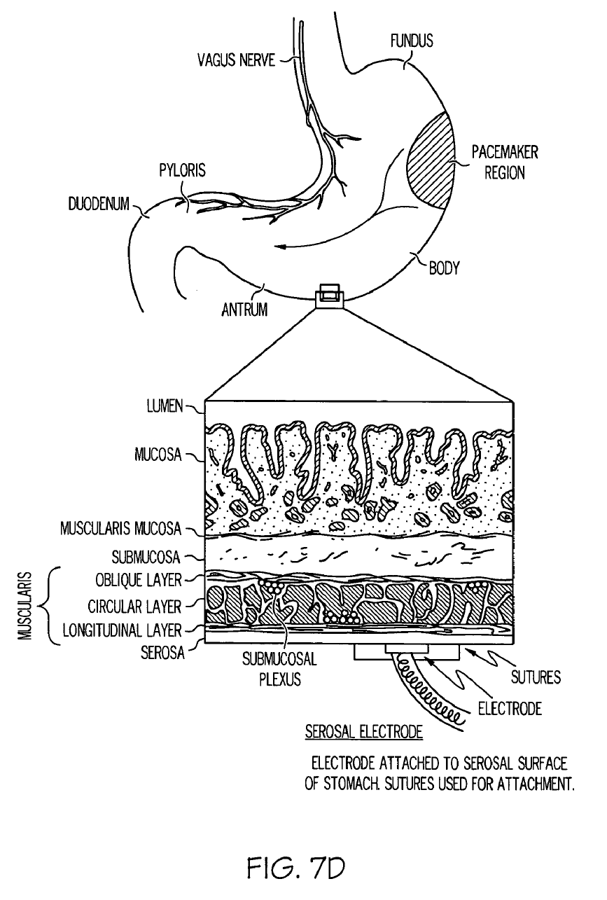

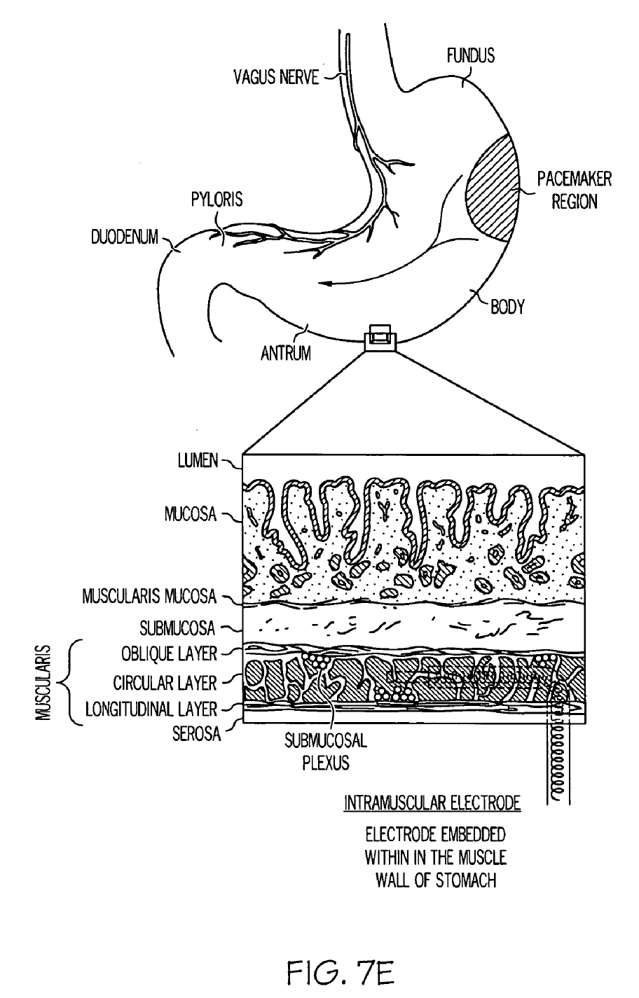

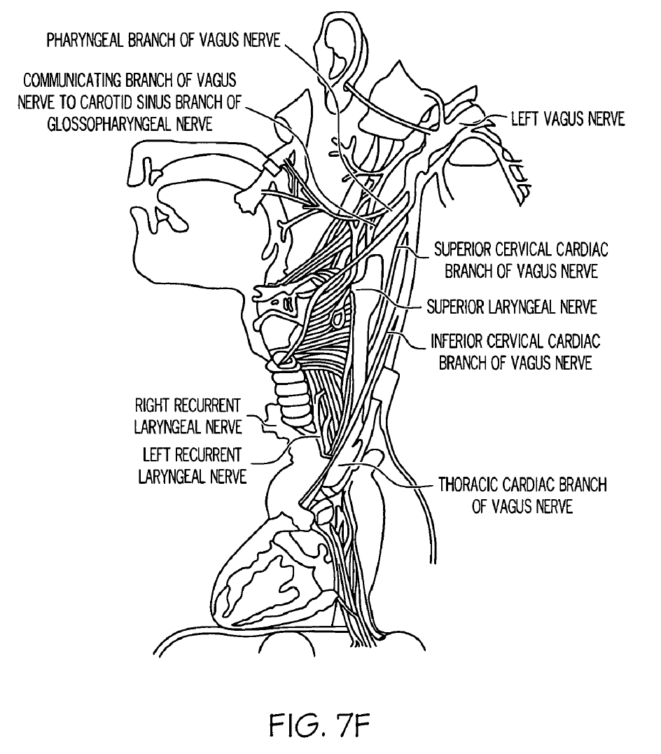

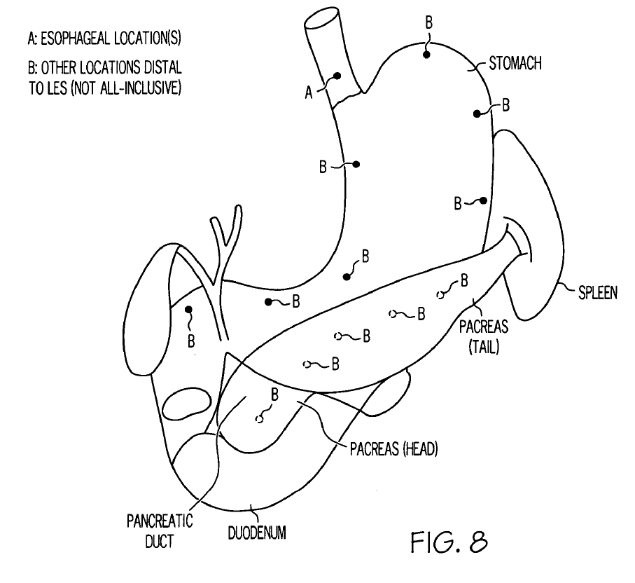

In accordance with several embodiments of the present invention, FIGS. 7a through 7f illustrate various locations for the placement of stimulation and sensing electrodes in and near the stomach. Electrodes 20 through 24 are placed in electrical contact or in proximity to target tissue 8. The electrode location may be selected based upon the obtained innervation of the vagus nerve and digestive system, the selected location's suitability for electrode connection, and the degree to which the location proves efficacious for treating a gastrointestinal disorder in a particular patient. Locations most suitable for electrode attachment and connection should be easily accessible by surgical or endoscopic means, and further be sufficiently mechanically robust and substantial to secure and retain electrodes 20-24 of leads 16 and/or 18.

Some specific electrode locations that are well innervated, and surgically or endoscopically accessible include, but are not limited to: (a) the plexus on the anterior superior and/or the anterior inferior pancreaticoduodenal arteries; (b) the plexus on the inferior pancreaticoduodenal artery; (c) the plexus on the jejunal artery; (d) the superior mesenteric artery and plexus; (d) the plexus on the gastroepiploic arteries; (e) the celiac ganglia and plexus; (f) the splenic artery and plexus; (g) the left lesser thoracic splanchic nerve; (h) the left greater thoracic splanchic nerve; (i) the principal anterior gastric branch of the anterior vagal trunk; (j) the left gastric artery and plexus; (k) the celiac branch of the anterior vagal trunk; (l) the anterior vagal trunk; (m) proximal, distal or portions between the proximal and distal portions of the vagus nerve; (n) the hepatic branch of the anterior vagal trunk; (o) the right and/or left inferior phrenic arteries and plexus; (p) the anterior posterior layers of the lesser omenium; (q) the branch from the hepatic plexus to the cardia via the lesser omenium; (r) the right greater thoracic splanchic nerve; (s) the vagal branch from the hepatic plexus to the pylorus; (t) the right gastric artery and plexus. Note that as discussed above, it is contemplated in the present invention that multiple leads be employed.

FIG. 8 illustrates some of the various locations in or near the stomach and/or vagus nerve of a patient for placing feedback control sensors according to some embodiments of closed-loop feedback control systems of the present invention.

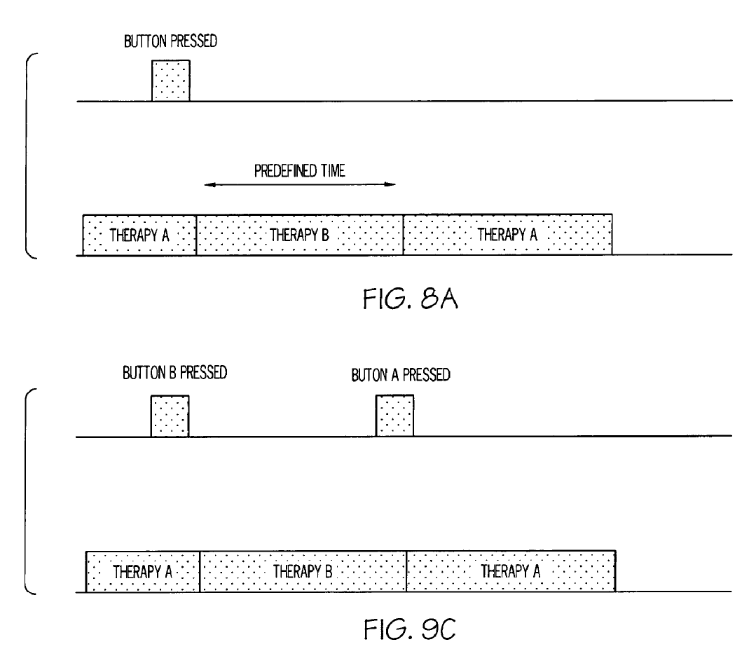

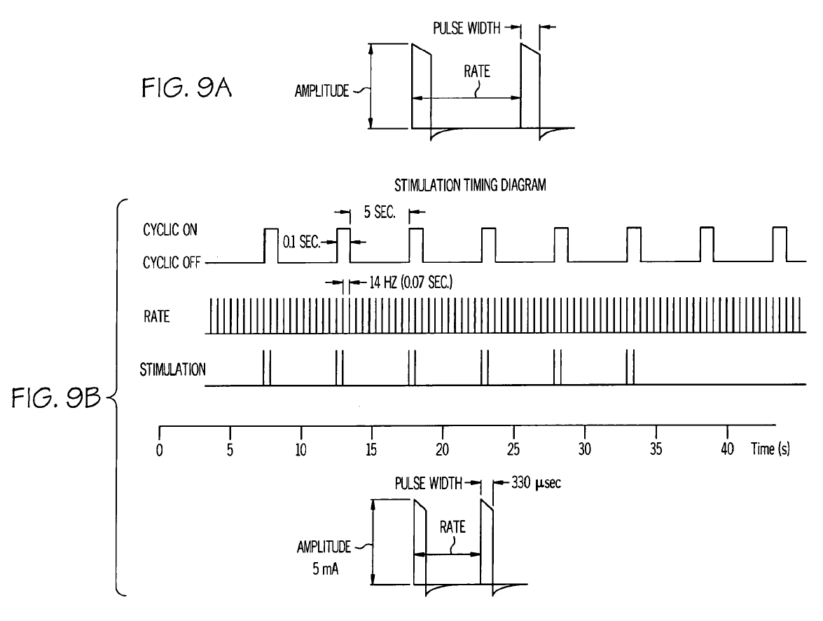

FIGS. 9a through 9c illustrate various representative electrical stimulation pulse, regime and control parameters according to some embodiments of the present invention. FIG. 9a illustrates a typical charge balanced square pulse used in many implantable electrical stimulation systems. As shown, amplitude, pulse width, and pulse rate are adjustable. In addition, the location at which an electrical pulse is applied may be changed by, e.g., administering the electrical pulse via an electrode located at a different location with the digestive system.

FIG. 9b shows a timing diagram illustrating the output of IPG 10 when the output signal provided thereby successively gated on and off. In FIG. 9b, IPG 10 is set to a frequency of 14 pulses per second, but is gated on for 0.1 seconds, and off for 5 seconds, resulting in an output of two pulses every five seconds. The on and off gating periods may be adjusted over a wide range.

In the present invention, electrical stimulation signal parameters may be selected to influence gastric acid secretion through direct stimulation of a target digestive tissue 8. The electrical stimulation signal is preferably charge-balanced for biocompatibility, and adapted to treat a gastrointestinal disorder. In the event multiple signals are employed to stimulate a desired site, the spatial and/or temporal phase between the signals may be adjusted or varied to produce the desired stimulation pattern or sequence. That is, in the present invention beam forming and specific site targeting via electrode array adjustments are contemplated. Examples of lead and electrode arrays and configurations that may be adapted for use in some embodiments of the present invention so as to better steer, control or target electrical stimulation signals provided thereby in respect of space and/or time include those disclosed in U.S. Pat. No. 5,501,703 to Holsheimer; U.S. Pat. No. 5,643,330 to Holsheimer; U.S. Pat. No. 5,800,465 to Thompson; U.S. Pat. No. 6,421,566 to Holsheimer; and U.S. Patent Application Publication No. 20020128694A1 to Holsheimer.

Representative ranges of electrical pulse stimulation parameters capable of being delivered by IPG 10 through leads 16 and 18 include the following:
Frequency: Between about 50 Hz and about 100 Hz;
   Between about 10 Hz and about 250 Hz; and
   Between about 0.5 Hz and about 500 Hz.
Amplitude: Between about 1 Volt and about 10 Volts;
   Between about 0.5 Volts and about 20 Volts; and
   Between about 0.1 Volts and about 50 Volts.
Pulse Width: Between about 180 microseconds and about 450 microseconds;
   Between about 100 microseconds and about 1000 microseconds;
   Between about 10 microseconds and about 5000 microseconds.

Further exemplary stimulation parameters of the system of the present invention include:
(a) A stimulation signal frequency ranging between:
   (i) about 0.10 to about 18,000 pulses per minute;
   (ii) about 1 to about 5,000 pulses per minute;
   (iii) about 1 to about 1,000 pulses per minute;
   (iv) about 1 to about 100 pulses per minute;
   (v) about 3 to about 25 pulses per minute;
(b) A stimulation signal pulse width ranging between:
   (i) about 0.01 mS to about 500 mS;
   (ii) about 0.1 mS to about 100 mS;
   (iii) about 0.1 mS to about 10 mS;
   (iv) about 0.1 mS to about 1 mS;
(c) A stimulation signal current ranging between:
   (i) about 0.01 mA to about 500 mA;
   (ii) about 0.1 mA to about 100 mA;
   (iii) about 0.1 mA to about 10 mA;
   (iv) about 1 mA to 100 mA, and
   (v) about 1 to about 10 mA.
(d) A stimulation signal which occurs continuously in accordance with the parameters of (a), (b), and (c) above, or a combination thereof;
(e) A stimulation signal which occurs discontinuously when the system turns on and off, where on and off are defined as a cycle time which may vary between about 1 second and about 60 seconds (for example, on=0.1 seconds, and off=5 seconds; on=1.0 sec and off=4 seconds, and so on; see FIGS. 9b and 9c).
(f) Stimulation signals having morphologies best characterized as (i) spikes, (ii) sinusoidal waves, or (iii) square pulses;

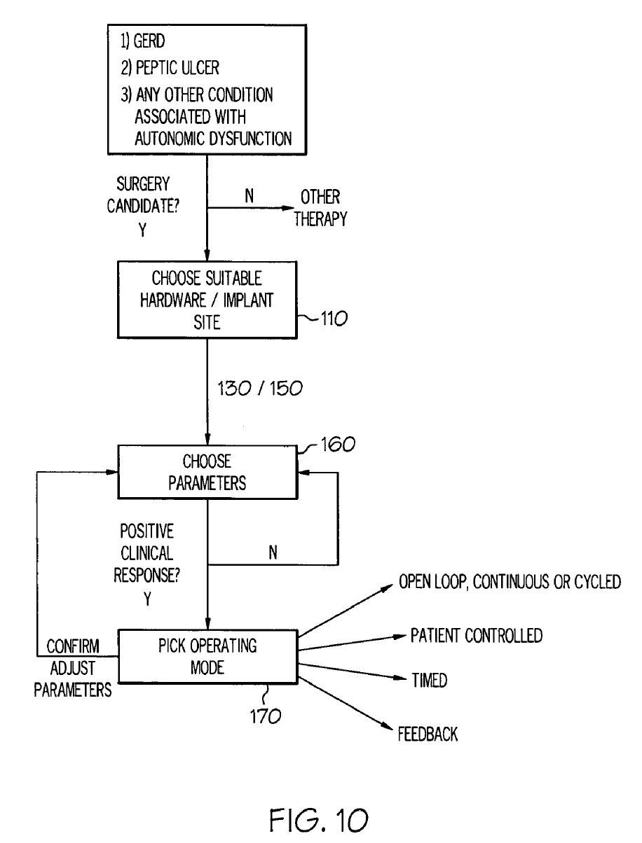

FIG. 10 illustrates several methods of stimulating a patient's digestive system or portion thereof so as to treat a gastrointestinal disorder in a subject. In FIG. 10, step 110 is employed to determine one or more desired stimulation locations (as illustrated in FIG. 5a through 5d and FIGS. 7a through 7f) positioned near or at one or more target locations 8 in the subject's digestive system. Step 130 is employed to implant IPG 10 in an appropriate location within the patient such that the proximal end of lead 16 may be operably connected thereto and such that IPG 10 is placed in such a location that discomfort and the risk of infection to the patient are minimized. Next IPG 10 is operably connected to lead 16, which may or may not require the use of optional lead extension 15 and lead connector 13. In Step 150, IPG 10 is activated and stimulation pulses are delivered to electrodes 20, 21, . . . n through lead 16 to the desired target stimulation location. In step 160, the electrical pulse stimulation parameters are adjusted to optimize the therapy delivered to the patient. Such adjustment may entail one or more of adjusting the number or configuration of electrodes or leads used to stimulate the selected location, pulse amplitude, pulse frequency, pulse width, pulse morphology (e.g., square wave, triangle wave, sinusoid, biphasic pulse, tri-phasic pulse, etc.), times of day or night when pulses are delivered, pulse cycling times, the positioning of the lead or leads, and/or the enablement or disablement of “soft start” or ramp functions respecting the stimulation regime to be provided. In step 170 the operating mode of the implanted system is selected. Optionally, parameters selected in step 160 may be adjusted after the operating mode has been selected to optimize therapy.

According to other embodiments of the present invention, implantable sensors and/or stimulation modules or leads may be implanted in desired portions of the gastro-intestinal tract by means of a vacuum-operated device which is endoscopically or otherwise emplaced within the gastrointestinal tract, followed by a portion of the tract being sucked up into a receiving chamber of the device, and the sensor, module or lead being implanted within the tissue held within the receiving chamber. See, for example, U.S. Pat. No. 6,098,629 for “Submucosal Esophageal Bulking Device” to Johnson et al.; U.S. Pat. No. 6,338,345 for “Submucosal Prosthesis Delivery Device” to Johnson et al.; U.S. Pat. No. 6,401,718 for “Submucosal Prosthesis Delivery Device” to Johnson et al.; and PCT Patent Application WO 02087657 for “Gastric Device and Suction Assisted Method for Implanting a Device on a Stomach Wall” assigned to Intrapace, Inc.

In still further embodiments of the present invention, various components of the gastro-intestinal electrical stimulation system may be extended, miniaturized, rendered wireless, powered, recharged or modularized into separate or discrete components in accordance with the teachings of, by way of example: U.S. Pat. No. 5,193,539 for “Implantable Microstimulator” to Schulman et al.; U.S. Pat. No. 5,193,540 for “Structure and Method of Manufacture of an Implantable Microstimulator” to Schulman et al.; U.S. Pat. No. 5,324,316 for “Implantable Microstimulators” to Schulman et al.; U.S. Pat. No. 5,358,514 for “Implantable Microdevice With Self-Attaching Electrodes” to Schulman et al.; U.S. Pat. No. 5,405,367 for “Structure and Method of Manufacture of an Implantable Microstimulator” to Schulman et al.; U.S. Pat. No. 5,957,958 for “Implantable Electrode Arrays” to Schulman et al.; U.S. Pat. No. 5,999,848 for “Daisy Chainable Sensors and Stimulators for Implantation in Living Tissue” to Gord et al.; U.S. Pat. No. 6,051,017 for “Implantable Microstimulator and Systems Employing the Same” to Loeb et al.; U.S. Pat. No. 6,067,474 for “Implantable Device With Improved Battery Recharging and Powering Configuration” to Schulman et al.; U.S. Pat. No. 6,205,361 for “Implantable Expandable Multicontact Electrodes” to Kuzma et al.; U.S. Pat. No. 6,212,431 for “Power Transfer Circuit for Implanted Devices” to Hahn et al.; U.S. Pat. No. 6,214,032 for “System for Implanting a Microstimulator” to Loeb; U.S. Pat. No. 6,315,721 for “System of Implantable Devices for Monitoring and/or Affecting Body Parameters” to Schulman et al.; U.S. Pat. No. 6,393,325 for “Directional Programming for Implantable Electrode Arrays” to Mann et al.; U.S. Pat. No. 6,516,227 for “Rechargeable Spinal Cord Stimulator System” to Meadows et al.

According to an embodiment, the invention provides a method for selecting candidate patients for digestive stimulation therapy. The method comprises selecting a patient suffering from or at risk of a gastrointestinal disorder. The method may further comprise determining whether the patient has a dysfunction of their autonomic nervous system. Those patients having an autonomic dysfunction are selected as candidates for digestive stimulation therapy. To determine whether the patient has an autonomic dysfunction, one or more of the indicators of autonomic function discussed above may be measured. A determination of whether the measured indicator falls with a range indicative of autonomic dysfunction may then be made. A range indicative of autonomic dysfunction may be a range that is considered abnormal, as discussed above. Alternatively, the range may a range preselected as being indicative of autonomic dysfunction, regardless of whether the measured indicator falls within a normal range.

It will also be recognized that combinations of various indicators falling individually within normal ranges may collectively be indicative of autonomic dysfunction.

According to various embodiments, the invention provides a method for treating a gastrointestinal (GI) disorder in a patient. Any GI disorder may be treated according to various embodiments of the invention discussed herein. Preferably, the GI disorder is a disorder associated with autonomic dysfunction. Most GI disorders, such as eating, motility, and endocrine disorders, including but not limited to obesity, gastro-esophageal reflux disease, constipation, heartburn, and gastroparesis, may be associated with autonomic dysfunction. Stimulation therapy directed to any one or more portion of a patient's digestive system may be efficacious for treatment of a GI disorder. Exemplary locations described above may prove to be particularly efficacious. One of skill in the art will recognize that the location(s) stimulated may be changed to accommodate the disease to be treated.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like are meant to include methods to alleviate, slow the progression, prevent, attenuate, or cure the targeted disease.

As used herein, “digestive system” means the cells tissues and organs involved in digesting food, such as stomach, duodenum, intestine, and pancreas. Nerves innervating digestive organs, such as those of the enteric nervous system and the vagus nerve, are also considered part of the digestive system.

As used herein, “gastrointestinal disorder” means a disease or disorder of the stomach duodenum, intestine, pancreas, and/or the like, or one or more portion thereof. Non-limiting examples gastrointestinal disorders include those disorders discussed herein-above.

The preceding specific embodiments are illustrative of the practice of the invention. It is to be understood, therefore, that other expedients known to those skilled in the art or disclosed herein may be employed without departing from the invention or the scope of the appended claims. For example, the present invention is not limited to the use of any particular specific configuration of an INS, leads or electrodes shown explicitly in the drawings hereof. Those skilled in the art will understand immediately that many variations and permutations of known implantable devices may be employed successfully in the present invention.

In the claims, means plus function clauses are intended to cover the structures described herein as performing the recited function and their equivalents. Means plus function clauses in the claims are not intended to be limited to structural equivalents only, but are also intended to include structures which function equivalently in the environment of the claimed combination. All printed publications and patents referenced hereinabove are hereby incorporated by referenced herein, each in its respective entirety.

What is claimed is:

1. A method for identifying a candidate patient for digestive stimulation therapy, comprising:
selecting a patient suffering from or at risk of a gastrointestinal disorder;
detecting cardiac activity of the patient; and
determining whether the detected cardiac activity is indicative of autonomic dysfunction,
wherein the patient is identified as a candidate for digestive stimulation therapy if the detected cardiac activity is indicative of autonomic dysfunction.

2. The method of claim 1, wherein detecting cardiac activity comprises detecting heart rate variability.

3. The method of claim 2, wherein detecting heart rate variability comprises analyzing a power spectrum of cardiac electrical activity.

4. The method of claim 3, wherein analyzing a power spectrum of cardiac electrical activity comprises analyzing the spectrum in a low frequency range of between about 0.04 Hz to about 0.15 Hz.

5. The method of claim 4, wherein analyzing a power spectrum of cardiac electrical activity further comprises analyzing the spectrum in a high frequency range of between about 0.18 Hz to about 0.4 Hz.

6. The method of claim 5, wherein analyzing a power spectrum of cardiac electrical activity comprises comparing power of the low frequency range to power of the high frequency range.

7. The method of claim 3, wherein analyzing a power spectrum of cardiac electrical activity comprises analyzing the spectrum in a high frequency range of between about 0.18 Hz to about 0.4 Hz.

8. The method of claim 2, wherein detecting heart rate variability comprises determining a standard deviation of mean R-R intervals.

9. The method of claim 1, further comprising:
applying a stimulation signal directly to at least a portion of a digestive system; and
redetecting the cardiac activity after applying the stimulation signal;
wherein determining whether the detected cardiac activity is indicative of autonomic dysfunction comprises determining whether the redected cardiac activity is indicative of an improvement in autonomic nervous system function, and
wherein the patient is identified as a candidate for digestive stimulation therapy if the redetected cardiac activity is indicative of an improvement in autonomic nervous system function.

10. The method of claim 9, wherein the stimulation signal is applied directly to the stomach.

11. The method of claim 9, wherein the stimulation signal is applied directly to the stomach pacemaker region.

12. The method of claim 9, wherein the stimulation is applied directly to a muscle wall of the stomach.

13. The method of claim 12, wherein the stimulation is applied to an oblique layer, a circular layer or a longitudinal layer of the muscular wall of the stomach.

14. The method of claim 9, wherein the stimulation is applied directly to one or more of a serosa layer, a myenteric plexus, and a submucosal plexus of the stomach.

15. The method of claim 9, wherein the stimulation signal is applied directly to the duodenum.

16. The method of claim 9, wherein the stimulation signal is applied directly to the intestine.

17. The method of claim 9, wherein the stimulation signal is applied directly to the pancreas.
